(19) United States
(12) Patent Application Publication
(10) Pub. No.: US 2022/0034875 A1
(43) Pub. Date: Feb. 3, 2022

(54) PARTICLE, AFFINITY PARTICLE, TEST REAGENT, AND DETECTION METHOD

(71) Applicant: CANON KABUSHIKI KAISHA, Tokyo (JP)

(72) Inventors: Ryo Natori, Tokyo (JP); Motokazu Kobayashi, Kanagawa (JP); Norishige Kakegawa, Tokyo (JP); Fumio Yamauchi, Kanagawa (JP); Kengo Kanazaki, Kanagawa (JP); Daisuke Sasaguri, Kanagawa (JP)

(21) Appl. No.: 17/502,629

(22) Filed: Oct. 15, 2021

Related U.S. Application Data
(63) Continuation of application No. PCT/JP2020/017278, filed on Apr. 22, 2020.

(30) Foreign Application Priority Data
Apr. 26, 2019 (JP) 2019-085963

(51) Int. Cl. G01N 33/543; C08L 101/02; G01N 33/553

(52) U.S. Cl. CPC G01N 33/54326; G01N 33/553; C08L 101/02

(57) ABSTRACT

Provided is a magnetic particle excellent in detection speed when a substance to be measured, such as an antigen or an antibody, is detected from a specimen. The particle includes a magnetic particle containing a magnetic material, wherein the magnetic particle has a resin on a surface, wherein the particle has a volume average particle diameter of 0.4 μm or more and 1.5 μm or less, wherein the particle has a density of 5.1 g/cm³ or more and 10.0 g/cm³ or less, and wherein the resin has a functional group capable of binding a ligand.

### CROSS-REFERENCE TO RELATED APPLICATIONS

This application is a Continuation of International Patent Application No. PCT/JP2020/017278, filed Apr. 22, 2020, which claims the benefit of Japanese Patent Application No. 2019-085963, filed Apr. 26, 2019, both of which are hereby incorporated by reference herein in their entirety.

### BACKGROUND OF THE INVENTION

### Field of the Invention

The present invention relates to a particle, an affinity particle, a test reagent, and a detection method.

### Description of the Related Art

In recent years, a magnetic particle has been used for a wide variety of applications. In particular, in the medical field, the magnetic particle is used for a specimen test for measuring an antigen, an antibody, or the like in blood and using the results for diagnosis. Specifically, there is given a method of detecting an antigen (antibody) from a specimen through use of a magnetic particle having an antibody (antigen), which specifically binds to the antigen (antibody), bound thereto and a flat plate having an antibody (antigen), which specifically binds to the antigen (antibody), immobilized thereon. When an antigen (antibody) is present in a specimen, the magnetic particle binds to the flat plate having the antibody (antigen) immobilized thereon via the antigen (antibody) through an antigen-antibody reaction. In such method of detecting an antigen (antibody), there is a demand for reduction in time up to detection (that is, a high detection speed).

As a particle to be used for a specimen test, there has been proposed a resin fine particle containing a magnetic material in which magnetite serving as a magnetic material is used (Japanese Patent Application Laid-Open No. 2004-099844). In addition, there has been proposed a composite particle containing a solid fine particle containing magnetite and a polymer compound (Japanese Patent Application Laid-Open No. 2009-300239). Further, there has been proposed a magnetic marker particle containing a magnetic particle and a polymer adhering to the surface of the magnetic particle (Japanese Patent Application Laid-Open No. 2012-177691).

The inventors of the present invention have made investigations of the detection of an antigen from a specimen through use of the particles described in Japanese Patent Application Laid-Open No. 2004-099844, Japanese Patent Application Laid-Open No. 2009-300239, and Japanese Patent Application Laid-Open No. 2012-177691, each having an antibody, which specifically binds to an antigen, immobilized thereon and a flat plate having an antibody, which specifically binds to the antigen, immobilized thereon, and as a result, have found that a detection speed may not be sufficiently obtained.

Accordingly, an object of the present invention is to provide a particle excellent in detection speed when a substance to be measured, such as an antigen or an antibody, is detected from a specimen. In addition, another object of the present invention is to provide an affinity particle, a test reagent, and a detection method each using the particle.

### SUMMARY OF THE INVENTION

The present invention relates to a particle including a magnetic particle containing a magnetic material, wherein the magnetic particle has a resin on a surface, wherein the particle has a volume average particle diameter of 0.4 μm or more and 1.5 μm or less, wherein the particle has a density of 5.1 g/cm³ or more and 10.0 g/cm³ or less, and wherein the resin has a functional group capable of binding a ligand.

The present invention also relates to an affinity particle including the particles having the above-mentioned configuration and a ligand that binds to the particle.

The present invention also relates to a test reagent including the affinity particle having the above-mentioned configuration and a dispersion medium for dispersing the affinity particle.

The present invention also relates to a method of detecting a substance to be measured contained in a specimen including: a first step of adding a specimen containing a substance to be measured and a test reagent containing an affinity particle and a dispersion medium for dispersing the affinity particle into a container having a first ligand immobilized on a lower side in a gravity direction; a second step of applying a magnetic field so that the affinity particle binds to the first ligand via the substance to be measured; and a third step of applying a magnetic field so that the affinity particle free from binding to the first ligand via the substance to be measured is separated away from the first ligand, wherein the affinity particle includes the particle having the above-mentioned configuration and a second ligand that binds to the particle, and wherein the first ligand and the second ligand are each bound to the substance to be measured.

Further features of the present invention will become apparent from the following description of exemplary embodiments.

### DESCRIPTION OF THE EMBODIMENTS

Various physical property values are values at 25° C. unless otherwise stated. The volume average particle diameter and density of a particle refer to a volume average particle diameter and a density also in consideration of a resin present on the surface of a magnetic particle. Now, as a substance to be measured to be detected from a specimen, an antigen is described as an example.

In the case where an antigen is detected from a specimen, when a particle having an antibody, which specifically binds to the antigen, bound thereto and a flat plate having an antibody, which specifically binds to the antigen, immobilized thereon are used, the flat plate is arranged on a lower side in a gravity direction, and a magnet or the like for generating a magnetic force is arranged in the vicinity of the flat plate. With this configuration, a magnetic particle can be attracted to the vicinity of the flat plate through action of gravity force and magnetic force. Then, through use of an antigen-antibody reaction, the particle attracted to the vicinity of the flat plate binds to the flat plate via the antibody and the antigen. Further, the particle free from binding to the flat plate is removed by arranging a magnet or the like on an upper side in the gravity direction. With this configuration, the presence of the particle binding to the flat plate can be recognized, and the antigen can be detected from the specimen.

When the substance to be measured, such as an antigen or an antibody, is detected from the specimen by the above-mentioned method, it is important to suppress the adsorption of proteins other than the substance to be measured to the magnetic particle to improve detection sensitivity. For this purpose, a resin having a functional group capable of binding a ligand is formed on the surface of the particle.

However, the following was found. Even when such particle is used, the gravity force and magnetic force applied to the particle are not sufficient. Accordingly, it takes time to attract the particle to the vicinity of the flat plate, with the result that the detection speed may not be sufficiently obtained.

In view of the foregoing, the inventors of the present invention have conceived that it is important to increase the volume average particle diameter and the density of the particle in order to make the gravity force and magnetic force applied to the particle sufficient. When the volume average particle diameter of the particle is increased, the gravity force and magnetic force applied to the particle can be made sufficient. When the density of the particle is increased, the magnetic force applied to the particle can be made sufficient. As a result, the moving speed of the particle is increased, and the detection speed is sufficiently obtained. Meanwhile, when the volume average particle diameter and density of the particle do not satisfy the predetermined ranges, the gravity force and magnetic force applied to the particle are not sufficient. Accordingly, it takes time to attract the particle to the vicinity of the flat plate, with the result that the detection speed is not sufficiently obtained.

The particle described in Japanese Patent Application Laid-Open No. 2004-099844 is formed by dispersing a magnetite fine particle of a nanometer size in an oily monomer and polymerizing the mixture by miniemulsion polymerization. The density of the particle is 1.3 g/cm³, and the magnetic force applied to the particle is not sufficient. Accordingly, it is conceived that it takes time to attract the particle to the vicinity of the flat plate, with the result that the detection speed is not sufficiently obtained.

The particle described in Japanese Patent Application Laid-Open No. 2009-300239 is produced by shearing a magnetic material dispersed in an organic solvent. The volume average particle diameter of the particle that can be produced in this manner is limited to about 0.3 μm. Because of this, the gravity force and magnetic force applied to the particle are not sufficient. Accordingly, it is conceived that it takes time to attract the particle to the vicinity of the flat plate, with the result that the detection speed cannot be sufficiently obtained.

The density of the magnetic marker particle described in Japanese Patent Application Laid-Open No. 2012-177691 is as high as 4.73 g/cm³. Accordingly, it is conceived that it takes time to attract the magnetic particle to the vicinity of the flat plate, with the result that the detection speed is not sufficiently obtained. In addition, the content of the magnetic material in the magnetic particle is less than 100 mass %, and hence saturation magnetization is small. Similarly, it is conceived that it takes time to attract the magnetic particle.

<Particle>

It is preferred that the particle be used for a specimen test. The particle includes a magnetic particle containing a magnetic material. In the present invention, the region of the magnetic particle in the particle is defined as a region specified by observation with a transmission electron microscope (TEM). An image is taken with the TEM at a magnification that allows about 20 particles to enter the field of view. Here, the TEM can photograph components having different specific gravities with contrast, and hence the region of the magnetic material in the magnetic particle can be identified. For the obtained image, the region of a circle obtained by connecting the magnetic material on an outermost side and the magnetic material, which is on the other end of the magnetic material on the outermost side and is also on the outermost side, with a straight line and drawing the circle so that the center of the straight line becomes the center of the circle is defined as the magnetic particle.

The content of the magnetic material in the magnetic particle is 80% or more and 100% or less, preferably 90% or more and 100% or less. When the content of the magnetic material in the magnetic particle is increased, the gravity force and magnetic force applied to the particle are increased, and the moving speed of the particle is further increased. As a result, the detection speed is improved. Here, the content of the magnetic material in the magnetic particle may be calculated by the following method.

The content of the magnetic material in the magnetic particle is determined by the expression: “content of magnetic material in particle×(average value of diameters of particles)³/(average value of diameters of magnetic particles)³”. The content and the average values in the expression are calculated as described below. For at least 20 particles, the diameter of each of the particles and the diameter of each of the magnetic particles determined as described above are measured through use of an image analyzer (Luzex AP, manufactured by Nireco Corporation), and the average values of the respective diameters are calculated. The content of the magnetic material in the particle is calculated through use of a thermogravimetric analyzer (TGA) or X-ray photoelectron spectroscopy (XPS). When the TGA is used, the content is determined based on the weight ratio before and after thermal decomposition of an organic component, and when the XPS is used, the content is determined based on the ratio of elements peculiar to the magnetic material.

The volume average particle diameter of the particle is 0.4 μm or more and 1.5 μm or less. When the volume average particle diameter is more than 1.5 μm, the detection speed is not sufficiently obtained, and in addition, the surface area per unit mass becomes small. Thus, the region in which the antigen-antibody reaction can be performed in the particle becomes small, and the efficiency of the antigen-antibody reaction may be decreased. The volume average particle diameter of the particle is preferably 0.7 μm or more and 1.2 μm or less, more preferably 0.7 μm or more and 0.9 μm or less. The volume average particle diameter may be measured by a dynamic light scattering method.

The density of the particle is 5.1 g/cm³ or more and 10.0 g/cm³ or less, more preferably 5.1 g/cm³ or more and 6.5 g/cm³ or less. The density may be measured with a dry automatic densitometer.

(Magnetic Material)

A magnetic material is a material that is magnetized by the application of a magnetic field. It is preferred that the magnetic material contain at least one kind selected from the group consisting of: a metal; and a metal oxide. Examples of the metal include iron, manganese, nickel, cobalt, and chromium. Examples of the metal oxide include triiron tetraoxide (Fe₃O₄), diiron trioxide (γ-Fe₂O₃), and ferrite. Of those, at least one kind selected from the group consisting of: iron; nickel; and magnetite is preferred as the magnetic material. Iron and nickel each have a large density, and magnetite has large saturation magnetization and small residual magnetization. Because of this, the gravity force and magnetic force applied to the magnetic particle are increased, and the detection speed is improved. Further, it is preferred that the magnetic material be iron or nickel.

Here, the magnetization refers to a phenomenon in which the magnetic material is polarized to become a magnet when an external magnetic field is applied to the magnetic material, and the saturation magnetization refers to a value at which the magnetization that is increased with the intensity of the magnetic field is saturated. Further, the residual magnetization refers to the magnetization that remains in the magnetic material when the magnetic field is eliminated after the external magnetic field is applied to the magnetic material.

When the magnetic material contains iron, the content of an iron atom in the magnetic material is preferably 80% or more and 100% or less because the density of the magnetic material can be improved. When the magnetic material contains nickel, the content of a nickel atom in the magnetic material is preferably 80% or more and 100% or less because the density of the magnetic material can be improved. Those contents each indicate the number of iron atoms or the number of nickel atoms (mol) with respect to the total number of atoms (mol) in the magnetic material.

It is preferred that the number of the magnetic materials in the magnetic particle be 1 or more. When the number of the magnetic materials in the magnetic particle is 2 or more, the particle diameter of each of the magnetic materials is preferably 50 nm or less, more preferably 5 nm or more and 30 nm or less. When the number of the magnetic materials in the magnetic particle is 1, the particle diameter of the magnetic material is preferably 0.1 μm or more, more preferably 1.2 μm or less. In particular, it is preferred that the number of the magnetic materials in the magnetic particle be 1, and the content of the magnetic material in the magnetic particle be 100%. That is, it is preferred that the magnetic particle be a single particle. Because of this, the density of the particle is increased, and the detection speed is improved.

In order to improve the detection speed of the particle, it is preferred to improve the sedimentation speed of the particle. As an expression for calculating the sedimentation speed of the particle, Stokes' law (V={g(ρₚ−ρ_{f})D²}/18μ) is known. V represents a sedimentation speed (cm/s), “g” represents a gravity acceleration rate (980.7 cm/s²), ρₚ represents the density (g/cm³) of the particle, and ρ_{f} represents the density (g/cm³) of a dispersion medium. In addition, D represents a particle diameter (cm), and ‘μ’ represents the viscosity (g/cm·s) of the dispersion medium. According to Stokes' law, the sedimentation speed V of the particle is increased in proportion to the square of the particle diameter.

The sedimentation speed determined by Stokes' law is preferably 1.0E-05 (cm/s) or more. When the sedimentation speed is less than 1.0E-05, it takes time to perform detection, and the detection speed may not be sufficiently obtained. The sedimentation speed is more preferably 5.0E-05 (cm/s) or more, still more preferably 1.0E-04 (cm/s) or more.

(Resin)

In the following description, the term “(meth)acrylate” means “acrylate or methacrylate.”

The resin has a functional group capable of binding a ligand. The functional group capable of binding a ligand is preferably at least one kind selected from the group consisting of: a carboxyl group; an amino group; a thiol group; an epoxy group; a maleimide group; and a succinimidyl group. Of those, a carboxy group is preferred as the functional group capable of binding a ligand.

In the polymerization of the resin, it is preferred to use: a monomer having a carboxy group, such as (meth)acrylic acid; a monomer having an amino group, such as (meth)acrylamide; a monomer having an epoxy group, such as glycidyl (meth)acrylate; and a monomer having a succinimidyl group, such as N-succinimidyl acrylate.

In the polymerization of the resin, in addition to the above-mentioned monomers, (meth)acrylates each having a hydrophilic group, such as glycerol (meth)acrylate, 2-hydroxyethyl (meth)acrylate, methoxyethyl (meth)acrylate, and polyethylene glycol mono(meth)acrylate; styrenes, such as styrene, p-chlorostyrene, and α-methylstyrene; and the like may also be used. In order to suppress non-specific adsorption to the magnetic particle, it is preferred to use (meth)acrylates each having a hydrophilic group.

In addition, the functional group capable of binding a ligand may also be added after the polymerization of the resin. For example, a thiol group may be introduced by adding a mercaptodiol to a resin obtained by polymerizing a monomer having a carboxyl group, such as (meth)acrylic acid. In addition, a maleimide group may be introduced by adding N-(2-hydroxyethyl) maleimide to the resin obtained by polymerization.

It is preferred that the resin have partial structures represented by the following formulae (2) and (3).

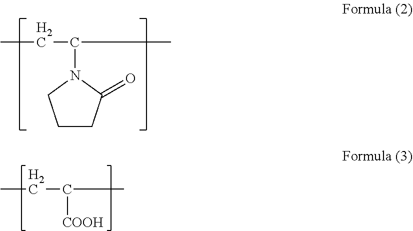

It is preferred that the resin have units derived from styrene and acrylic acid. Here, the unit means a unit structure corresponding to one monomer.

It is preferred that the resin have a siloxane bond. That is, a monomer containing an organic silane, such as vinyltrimethoxysilane, vinyltriethoxysilane, p-styryltrimethoxysilane, 3-methacryloxypropylmethyldimethoxysilane, 3-methacryloxypropyltrimethoxysilane, 3-methacryloxypropylmethyldiethoxysilane, 3-methacryloxypropyltriethoxysilane, or 3-acryloxypropyltrimethoxysilane, is preferably used in the polymerization of the resin.

The weight average molecular weight of the resin is preferably 10,000 or more, more preferably 100,000 or less. In addition, the weight average molecular weight of the resin is more preferably 40,000 or more and 100,000 or less, still more preferably 60,000 or more and 80,000 or less. The weight average molecular weight may be measured by gel permeation chromatography.

<Affinity Particle>

An affinity particle of the present invention includes the particle having the above-mentioned configuration and a ligand that binds to the particle. The ligand refers to a compound that specifically binds to a receptor of a substance to be measured. The ligand binds to the substance to be measured at a predetermined site and has selectively or specifically high affinity. Examples of a combination of the substance to be measured and the ligand include: an antigen and an antibody; an enzyme protein and a substrate thereof; and a signal substance, such as a hormone or a neurotransmitter, and a receptor thereof. It is preferred that the ligand be any one of an antigen and an antibody. In addition, one kind of ligand may be bound to the affinity particle, and a plurality of kinds of ligands may be bound to the affinity particle. Through use of the affinity particle having a plurality of kinds of ligands bound thereto, a plurality of kinds of substances to be measured can be easily detected. In addition, when there are a plurality of recognition sites at which the substance to be measured is recognized by the ligand, it is appropriate to bind a plurality of kinds of ligands corresponding to the plurality of recognition sites to the affinity particle.

In order to immobilize the ligand on the particle to form an affinity particle, a conventionally known method, such as chemical bonding or physical adsorption, may be applied through use of the functional group capable of binding a ligand of the particle. In particular, when the ligand is bound to the particle through an amide bond, a catalyst, such as 1-[3-(dimethylaminopropyl)-3-ethylcarbodiimide], may be used.

It is preferred that the affinity particle be used for a method involving binding the magnetic particle to a flat plate having a ligand immobilized thereon via a substance to be measured through an antigen-antibody reaction and removing an unbound particle from the vicinity of the flat plate with a magnetic force, to thereby detect the magnetic particle bound to the flat plate.

<Test Reagent>

A test reagent includes the affinity particle having the above-mentioned configuration and a dispersion medium for dispersing the affinity particle. The content of the affinity particle is preferably 0.001 mass % or more and 20 mass % or less, more preferably 0.01 mass % or more and 10 mass % or less assuming that the total mass of the dispersion medium is 100 mass %. The test reagent may contain a solvent, a blocking agent, and the like. Two or more kinds of solvents, blocking agents, and the like may be combined. Examples of the solvent include buffers, such as a phosphate buffer, a glycine buffer, a Good's buffer, a Tris buffer, and an ammonia buffer.

<Detection Method>

In this embodiment, a method of detecting a substance to be measured contained in a specimen includes at least the following steps.

(First Step)

In the first step, a specimen containing a substance to be measured and a test reagent containing an affinity particle and a dispersion medium for dispersing the affinity particle are added to a container having a first ligand immobilized on a lower side in a gravity direction.

(Second Step)

In the second step, a magnetic field is applied so that the affinity particle binds to the first ligand via the substance to be measured.

(Third Step)

In the third step, a magnetic field is applied so that the affinity particle free from binding to the first ligand via the substance to be measured is separated away from the first ligand.

In the detection method according to this embodiment, it is only required that the first ligand and the second be each capable of binding to the substance to be measured, and the above-mentioned ligand may be used. The first ligand and the second ligand may be identical to or different from each other.

In addition, the first ligand may be provided on a flat surface formed on the lower side in the gravity direction in the container (housing) or on a flat plate provided in the container. An example of the detection method includes: a first step of putting a specimen and the test reagent having the above-mentioned configuration in a container provided with a flat plate having a ligand, which binds to the affinity particle, immobilized thereon on the lower side in the gravity direction; a second step of arranging a magnet in the vicinity of the flat plate to attract the affinity particle in the test reagent to the vicinity of the flat plate; and a third step of arranging a magnet on an upper side in the gravity direction to remove the affinity particle free from binding to the flat plate. Further, through performance of a step of detecting a signal from the affinity particle bound to the first ligand via the substance to be measured, the presence or absence and concentration of the substance to be measured can be measured. The presence (presence or absence and concentration) of the substance to be measured in the specimen can be recognized when the particle binds to the flat plate via the substance to be measured in the specimen through an antigen-antibody reaction.

### EXAMPLES

The present invention is described in more detail below by way of Examples and Comparative Examples. The present invention is by no means limited to Examples below without departing from the gist of the present invention. “Part(s)” and “%” with regard to the description of the amounts of components are by mass, unless otherwise stated.

### Example 1

In a 300 mL flask, 100 mL of a 10 mM Tris buffer (manufactured by Tokyo Chemical Industry Co., Ltd.) and 1.0 g of iron particles (NP-FE-1, manufactured by EM Japan Co., Ltd.) serving as a magnetic material were mixed, and the mixture was stirred at 25° C. for 20 minutes. Here, the pH of the Tris buffer was adjusted with hydrochloric acid to be 9.0. In addition, the volume average particle diameter of the iron particles was 0.8 μm. 1.0 g of dopamine hydrochloride (manufactured by Sigma-Aldrich Japan) was added to the obtained solution, and the mixture was stirred at 25° C. overnight to obtain a dispersion liquid.

After that, an operation involving centrifuging the dispersion liquid, removing a supernatant, and washing the resultant with ion-exchanged water was performed three times, and a phosphate buffer (manufactured by Kishida Chemical Co., Ltd.) having a pH of 7.4 was added to a precipitate, to thereby obtain a dispersion liquid substituted with a phosphate buffer. 1.0 g of an N-vinylpyrrolidone-acrylic acid copolymer (manufactured by Polymer Source. Inc.) serving as a resin was dissolved in the obtained dispersion liquid. Here, the weight average molecular weight of the N-vinylpyrrolidone-acrylic acid copolymer was 60,000, and a ratio between the total molar quantity of a unit structure corresponding to vinylpyrrolidone and the total molar quantity of a unit structure corresponding to acrylic acid was 4:6.

0.4 g of 3-methacryloxypropyltrimethoxysilane (LS-3380, manufactured by Shin-Etsu Chemical Co., Ltd.) and 1.3 g of styrene (manufactured by Kishida Chemical Co., Ltd.) were added to the dispersion liquid containing the resin, and the resultant was stirred at 25° C. for 15 minutes while nitrogen was blown, to thereby obtain an emulsion. The obtained emulsion was heated to 70° C. through use of an oil bath, and a solution obtained by dissolving 0.3 g of potassium peroxodisulfate (manufactured by Wako Pure Chemical Industries, Ltd. (current name: Fujifilm Wako Pure Chemical Corporation)) in 10 mL of a phosphate buffer (manufactured by Kishida Chemical Co., Ltd.) having a pH of 7.4 was added to the heated emulsion. The emulsion was stirred at 70° C. for 7 hours, and then the temperature of the emulsion was returned to 25° C. After that, the emulsion was centrifuged, and the particles in which the resin was present on the surfaces of the magnetic particles were collected. A supernatant was discarded, and the particles were redispersed with ion-exchanged water. An operation involving collecting the particles by centrifugation and redispersing the particles with ion-exchanged water was performed three times to obtain a dispersion liquid of particles 1 having a particle content of 1.0%.

### Example 2

In the preparation of the dispersion liquid of the particles of Example 1, the materials to be added to the dispersion liquid containing the resin were changed to only styrene (manufactured by Kishida Chemical Co., Ltd.). Except for the foregoing, a dispersion liquid of particles 2 having a particle content of 1.0% was obtained by the same procedure as that in the preparation of the dispersion liquid of the particles of Example 1.

### Example 3

In a 300 mL flask, 100 mL of a 10 mM Tris buffer (manufactured by Tokyo Chemical Industry Co., Ltd.) and 1.0 g of iron particles (NP-FE-1, manufactured by EM Japan Co., Ltd.) serving as a magnetic material were mixed, and the mixture was stirred at 25° C. for 20 minutes. Here, the pH of the Tris buffer was adjusted with hydrochloric acid to be 9.0. In addition, the volume average particle diameter of the iron particles was 0.8 μm. 1.0 g of dopamine hydrochloride (manufactured by Sigma-Aldrich Japan) was added to the obtained solution, and the mixture was stirred at 25° C. overnight to obtain a dispersion liquid.

After that, an operation involving centrifuging the dispersion liquid, removing a supernatant, and washing the resultant with ion-exchanged water was performed three times, and ion-exchanged water was added to a precipitate, to thereby obtain a dispersion liquid substituted with ion-exchanged water. 1.0 g of polyvinylpyrrolidone (PVP-K30, manufactured by Kishida Chemical Co., Ltd.) and 2.0 g of 1-amino-3,6,9,12,15,18-hexaoxahenicosan-21-oic acid were mixed with the obtained dispersion liquid. The mixture was stirred at 25° C. overnight to obtain a dispersion liquid. The dispersion liquid was centrifuged, and the particles in which the resin was present on the surfaces of the magnetic particles were collected. A supernatant was discarded, and the particles were redispersed with ion-exchanged water. An operation involving collecting the particles by centrifugation and redispersing the particles with ion-exchanged water was performed three times to obtain a dispersion liquid of particles 3 having a particle content of 1.0%. The weight average molecular weight of the resin was 60,000.

### Example 4

750 g of an aqueous solution containing 0.5% of sodium dodecylbenzenesulfonate and 0.5% of a nonionic emulsifier (Emulgen 150, manufactured by Kao Corporation) and 30 g of iron particles (NP-FE-1, manufactured by EM Japan Co., Ltd.) were serially put in a 1 L separable flask, to thereby obtain a solution. The obtained solution was dispersed with a homogenizer and heated to 70° C.

A dispersion liquid obtained by putting and dispersing 14 g of cyclohexyl methacrylate, 1 g of trimethylolpropane trimethacrylate, and 0.3 g of t-butyl peroxy-2-ethylhexanate (Perbutyl O, manufactured by Nippon Oil and Fats Co., Ltd.) in 75 g of an aqueous solution was added dropwise to the obtained solution over 1 hour. Here, the aqueous solution contained 0.5% of sodium dodecylbenzenesulfonate and 0.5% of a nonionic emulsifier (Emuigen 150, manufactured by Kao Corporation). As a result, a liquid containing particles having a first layer containing a resin on the surfaces of the magnetic particles was obtained.

A dispersion liquid obtained by putting and dispersing 14 g of cyclohexyl methacrylate, 1 g of trimethylolpropane trimethacrylate, and 0.3 g of t-butyl peroxy-2-ethylhexanate (Perbutyl O, manufactured by Nippon Oil and Fats Co., Ltd. (current name: NOF Corporation)) in 75 g of an aqueous solution was added dropwise to the obtained liquid over 1 hour. Here, the aqueous solution contained 0.5% of sodium dodecylbenzenesulfonate and 0.5% of a nonionic emulsifier (Emulgen 150, manufactured by Kao Corporation). As a result, a liquid containing particles in which two layers (first layer and second layer) containing a resin were present on the surfaces of the magnetic particles was obtained. The weight average molecular weight of the resin contained in the two layers was 80,000.

After the temperature of the obtained liquid was raised to 80° C., polymerization was continued for 2 hours to complete the reaction, to thereby obtain a dispersion liquid. The dispersion liquid was centrifuged, and the magnetic particles in which the resin was present on the surfaces of the magnetic particles were collected. A supernatant was discarded, and the particles were redispersed with ion-exchanged water. An operation involving collecting the particles by centrifugation and redispersing the particles with ion-exchanged water was performed three times to obtain a dispersion liquid of particles 4 having a particle content of 1.0%.

### Example 5

In the preparation of the dispersion liquid of the particles in Example 3, the kind of the magnetic material was changed to nickel particles. Here, the volume average particle diameter of the nickel particles was 0.45 μm. Except for the foregoing, a dispersion liquid of particles 5 having a particle content of 1.0% was obtained by the same procedure as that in the preparation of the dispersion liquid of the particles of Example 3.

### Comparative Example 1

In the preparation of the dispersion liquid of the particles of Example 3, the kind of the magnetic material was changed to magnetite particles. Here, the volume average particle diameter of the magnetite particles was 0.7 μm. Except for the foregoing, a dispersion liquid of particles 6 having a particle content of 1.0% was obtained by the same procedure as that in the preparation of the dispersion liquid of the particles of Example 3.

### Comparative Example 2

FeCl₃ and FeCl₂ were dissolved in water to prepare a solution. The solution was vigorously stirred while being maintained at 25° C. After that, ammonia water was added to the solution to obtain a suspension of magnetite. Oleic acid was added to the obtained suspension, and the suspension was stirred at 70° C. for 1 hour and at 110° C. for 1 hour to obtain a slurry. The slurry was washed with a large amount of water and dried under reduced pressure to obtain powdered hydrophobized magnetite. The average particle diameter of the obtained hydrophobized magnetite was 11 nm, and the molecular weight distribution thereof was 1.3. Here, the average particle diameter of the hydrophobized magnetite is a value calculated through use of a transmission electron microscope.

Next, 2 g of styrene and 3 g of hydrophobized magnetite were weighed in 4 g of chloroform to obtain a mixed solution 1. 0.01 g of sodium dodecyl sulfate was dissolved in 12 g of water to obtain a mixed solution 2. The mixed solution 1 and the mixed solution 2 were mixed to obtain a mixed solution 3, and the mixed solution 3 was sheared with a stirring homogenizer for 30 minutes to obtain a liquid containing magnetic particles each containing a plurality of magnetic materials.

Chloroform was preferentially fractionated from a dispersion medium by treating the liquid containing the magnetic particles with an evaporator under reduced pressure. After the obtained magnetic particles were deoxidized by nitrogen bubbling, 0.01 g of a polymerization initiator was added to the resultant, and styrene was polymerized at 70° C. for 6 hours. As a result, a dispersion liquid of particles 7 in which a resin was present on the surfaces of the magnetic particles (the content of the particles 7 was 1.0%) was obtained. As the polymerization initiator, 2,2′-azobis(2-methylpropionamidine) dihydrochloride was used. The weight average molecular weight of the resin was 70,000.

### Comparative Example 3

FeCl₃ and FeCl₂ were dissolved in water to prepare a solution. The solution was vigorously stirred while being maintained at 25° C. Then, ammonia water was added to the solution to obtain a suspension of magnetite. Oleic acid was added to the obtained suspension, and the suspension was stirred at 70° C. for 1 hour and at 110° C. for 1 hour to obtain a slurry. The slurry was washed with a large amount of water and dried under reduced pressure to obtain powdered hydrophobized magnetite.

An aqueous solution obtained by dissolving 0.3 g of a nonionic surfactant (Emulgen 1150S-70, manufactured by Kao Corporation) having a polyethylene oxide chain was added to the obtained hydrophobized magnetite, and the mixture was subjected to sonication. As a result, the nonionic surfactant was adsorbed to the surfaces of the hydrophobized magnetite particles to obtain a colloidal solution of magnetite particles having hydrophilicity imparted to the surfaces of the particles. An aqueous solution obtained by dissolving 10 μL of aminoundecane serving as an ionic surfactant in 56 μL of a HCl solution was added to the colloidal solution to obtain a colloidal solution of magnetite particles in which both the nonionic surfactant and the ionic surfactant were adsorbed to the surfaces of the particles.

2.7 g of styrene, 0.3 g of acrylic acid, 0.025 g of a polymerization initiator, 0.08 g of divinylbenzene, and 2.5 g of diethyl ether were added to the obtained colloidal solution, and the mixture was subjected to sonication, to thereby obtain an emulsion. Here, azobisisobutyronitrile was used as the polymerization initiator, and divinylbenzene was used as a crosslinking agent.

Water was added to the emulsion so that the total amount became 125 g, and the mixture was subjected to sonication. Then, the emulsion was heated under stirring at 350 rpm. When the temperature of the emulsion reached 70° C., an aqueous solution obtained by dissolving 50 mg of a water-soluble polymerization initiator (V-50, manufactured by Wako Pure Chemical Industries, Ltd. (current name: Fujifilm Wako Pure Chemical Corporation)) in 5 mL of pure water was added to the emulsion. After that, a polymerization reaction was performed for 12 hours to obtain a dispersion liquid of particles 10 in which the resin was present on the surfaces of the magnetic particles (particle content: 1.0%). The weight average molecular weight of the resin was 60,000.

[Content of Magnetic Material in Magnetic Particle]

The content of the magnetic material in the magnetic particle was determined from the expression “Content of magnetic material in particle×(average value of diameters of particles)³/(average value of diameters of magnetic particles)³”. The content and the average values in the expression were calculated as described below. For at least 20 particles, the diameter of each of the particles and the diameter of each of the magnetic particles were measured through use of an image analyzer (Luzex AP, manufactured by Nireco Corporation), and the average values of the respective diameters were calculated. The content of the magnetic material in the particle was calculated through use of X-ray photoelectron spectroscopy (XPS).

[Method of Measuring Volume Average Particle Diameter]

The volume average particle diameter of the particle is a value measured through use of a particle size distribution analyzer (Nanotrac UPA-EX150, manufactured by Nikkiso Co., Ltd.) by a dynamic light scattering method under the condition that an aqueous solution obtained by diluting a dispersion liquid of particles with pure water by 250 times (volume basis) was used as a measurement sample. The measurement conditions are as follows: SetZero: 30 s, number of times of measurement: 3 times, measurement time: 180 seconds, shape: non-spherical shape, and refractive index: 1.51.

[Method of Measuring Density]

The density of the magnetic particle is a value measured through use of a dry automatic densitometer (AccuPyc II 1340, manufactured by Shimadzu Corporation). The measurement was performed at a temperature of 23° C.

A measuring apparatus includes a sample chamber to which a helium gas introduction pipe is connected and an expansion chamber to which a helium gas discharge pipe is connected. In addition, the sample chamber and the expansion chamber are connected to each other through a coupling pipe. The helium gas introduction pipe, the coupling pipe, and the helium gas discharge pipe are each provided with a stop valve. The sample chamber has a pressure gauge for measuring the pressure in the chamber.

Through use of the above-mentioned measuring apparatus, the measurement was specifically performed as described below. First, a volume (V_{CELL}) of the sample chamber and a volume (V_{EXP}) of the expansion chamber were measured through use of a standard sphere. As a sample, a sample dried at 40° C. for 24 hours under reduced pressure was used. The pressure is a gauge pressure that is a pressure obtained by subtracting an ambient pressure from an absolute pressure. A sample was put in the sample chamber, and a helium gas was allowed to flow for 2 hours through the helium gas introduction pipe of the sample chamber, the coupling pipe, and the helium gas discharge pipe of the expansion chamber. Thus, the inside of the measuring apparatus was purged with the helium gas. Next, the stop valves of the coupling pipe and the helium gas discharge pipe were closed, and a helium gas was introduced into the sample chamber from the helium gas introduction pipe until the helium gas reached 134 kPa. Then, the stop valve of the helium gas introduction pipe was closed. A pressure (P₁) in the sample chamber was measured 5 minutes after closing the stop valve of the helium gas introduction pipe. Further, the stop valve of the coupling pipe was opened to transfer the helium gas to the expansion chamber, and a pressure (P₂) in this case was measured.

A volume (V_{SAMP}) of the sample was calculated from the following expression (4). Through use of the obtained value and a weight (W_{SAMP}) of the sample, a density ρ (W_{SAMP}/V_{SAMP}) of the sample was determined.

V_{SAMP}=V_{CELL}−V_{EXP}[(P₁−P₂)−1] Expression (4)

[Method of Measuring Weight Average Molecular Weight]

The weight average molecular weight of the resin was measured by gel permeation chromatography (GPC) as described below. The resin was dissolved in tetrahydrofuran (THF) at 25° C. over 24 hours. The obtained solution was filtered through a membrane filter to obtain a sample solution. The sample solution was adjusted so that the concentration of a component soluble in the THF was about 0.3%. Through use of the sample solution, the weight average molecular weight of the resin was measured under the following conditions.

Apparatus: Waters 2695 Separations Module, manufactured by Waters
RI detector: 2414 detector, manufactured by Waters
Column: KF-806M quadruple column, manufactured by Showa Denko K.K.
Eluent: tetrahydrofuran (THF)
Flow rate: 1.0 mL/min
Oven temperature: 40° C.
Sample injection amount: 100 μL

In the calculation of the weight average molecular weight of the resin, a molecular weight calibration curve prepared by using a standard polystyrene resin (TSK standard polystyrene F-850, F-450, F-288, F-128, F-80, F-40, F-20, F-10, F-4, F-2, F-1, A-5000, A-2500, A-1000, or A-500, manufactured by Tosoh Corporation) was used.

<Production of Affinity Particles>

An anti-CRP antibody was bound to each of the magnetic particles of Examples and Comparative Examples as described below. Here, the CRP is an abbreviation for a C-Reactive Protein, and refers to a protein that appears in the blood when inflammation occurs in a body, or a tissue is destroyed.

First, a dispersion liquid of the magnetic particles was centrifuged at 15,000 rpm (20,400 g) for 15 minutes to precipitate the magnetic particles. After a supernatant was removed, a pellet of the magnetic particles was redispersed with a IVIES buffer, and water-soluble carbodiimide (WSC) and N-hydroxysuccinimide were added. Then, the mixture was stirred at 25° C. for 30 minutes, and the magnetic particles were collected by centrifugation. The collected magnetic particles were washed with the IVIES buffer and redispersed with the MES buffer. An anti-CRP antibody was added to the resultant so that the final concentration of the antibody became 2 mg/mL. Then, the mixture was stirred at 25° C. for 60 minutes. The magnetic particles were collected by centrifugation and washed with a HEPES buffer to obtain a dispersion liquid of affinity particles having the anti-CRP antibody bound thereto.

The fact that the antibody was bound to each of the magnetic particles was recognized by a bicinchoninic acid (BCA) assay capable of colorimetrically quantifying the protein on the amount of decrease in concentration of the antibody in the MES buffer having the antibody added thereto.

[Recognition of Non-Specific Adsorptivity]

The non-specific adsorptivity of the affinity particles was recognized through use of the dispersion liquid of the affinity particles of the Examples. 51 μL of a serum solution diluted 50-fold with phosphate buffered saline was added to 50 μL of the dispersion liquid of the affinity particles, and before and after the addition, the presence or absence of particle aggregation caused by non-specific adsorption to the affinity particles was visually recognized. As a result, no significant aggregation was observed.

[Recognition of Detection Sensitivity]

The detection sensitivity of the affinity particles was recognized through use of the dispersion liquid of the affinity particles of the Examples. A flat plate having an anti-CRP antibody bound thereto was set in a lower portion of a 6 mL vial. In addition, 10 mL of an aqueous solution containing affinity particles at a content of 0.01% was put in the vial to disperse the affinity particles. 30 μL of an anti-CRP antigen was added to the vial, and a neodymium magnet was applied to the lower portion of the vial for 5 minutes. After that, the neodymium magnet was removed, and the vial was allowed to stand for 1 minute. Next, the neodymium magnet was applied to an upper portion of the vial for 1 minute to remove the affinity particles of a supernatant, and then the flat plate having the anti-CRP antibody bound thereto was taken out. It was recognized with a scanning electron microscope (SEM) that the affinity particles were bound to the antibody on the flat plate via the antigen.

<Evaluation>

In the present invention, 4 is defined as an acceptable level and 3, 2, or 1 is defined as an unacceptable level in the following evaluation criteria for evaluation. The evaluation results are shown in Table 1.

(Detection Speed)

10 mL of an aqueous solution containing affinity particles at a content of 0.01% was put in a vial to disperse the affinity particles. After a neodymium magnet was applied to a lower portion of the vial for 60 seconds, the neodymium magnet was removed, and the state in which the particles were settled in the vial was visually observed. Easy settling of the affinity particles means a high detection speed of the concentration of the settled affinity particles.

4: All affinity particles were settled in 120 seconds, and a supernatant was clear.

3: All affinity particles were settled in 120 seconds, but the lower part of the supernatant was turbid.

2: Affinity particles were partially settled in 120 seconds, and affinity particles were observed in a part of the supernatant.

1: Affinity particles were partially settled in 120 seconds, and affinity particles were observed in the entire supernatant.

TABLE 1

| | Volume average | | Content of | |
|---|---|---|---|---|
| | particle | | magnetic material | |
| | diameter D | Density ρ | in magnetic | Detection |
| | (μm) | (g/cm³) | particle (%) | speed |
| Example 1 | 0.9 | 6.4 | 100 | 4 |
| Example 2 | 0.9 | 6.4 | 100 | 4 |
| Example 3 | 0.9 | 5.8 | 100 | 4 |
| Example 4 | 0.9 | 5.1 | 100 | 4 |
| Example 5 | 0.7 | 6.5 | 100 | 4 |
| Comparative | 0.7 | 4.2 | 100 | 3 |
| Example 1 | | | | |
| Comparative | 0.3 | 3.2 | 85 | 2 |
| Example 2 | | | | |
| Comparative | 0.3 | 1.4 | 5 | 1 |
| Example 3 | | | | |

As described above, according to the present invention, there can be provided the particle excellent in detection speed when a substance to be measured, such as an antigen or an antibody, is detected from a specimen, and the affinity particle, the test reagent, and the detection method each using the particles.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A particle comprising a magnetic particle containing a magnetic material,
wherein the magnetic particle has a resin on a surface,
wherein the particle has a volume average particle diameter of 0.4 μm or more and 1.5 μm or less,
wherein the particle has a density of 5.1 g/cm³ or more and 10.0 g/cm³ or less, and
wherein the resin has a functional group capable of binding a ligand.

2. The particle according to claim 1, wherein the particle has a density of 5.1 g/cm³ or more and 6.5 g/cm³ or less.

3. The particle according to claim 1, wherein the particle has a volume average particle diameter of 0.7 μm or more and 1.2 μm or less.

4. The particle according to claim 1, wherein the particle has a volume average particle diameter of 0.7 μm or more and 0.9 μm or less.

5. The particle according to claim 1, wherein the resin has a weight average molecular weight of 10,000 or more.

6. The particle according to claim 1, wherein the resin has repeating units represented by the following formulae (2) and (3).

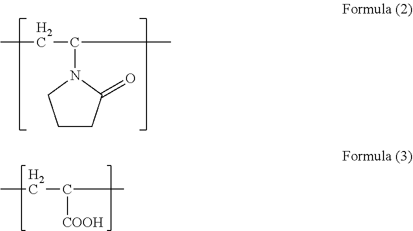

7. The particle according to claim 1, wherein the resin has units derived from styrene and acrylic acid.

8. The particle according to claim 1, wherein the particle is used for a specimen test.

9. The particle according to claim 1,
wherein a number of the magnetic materials in the magnetic particle is 1, and
wherein the magnetic particle contains the magnetic material at a content of 100%.

10. The particle according to claim 1, wherein the magnetic material contains at least one kind selected from the group consisting of: a metal; and a metal oxide.

11. The particle according to claim 1, wherein the magnetic material is at least one kind selected from the group consisting of: iron; nickel; and magnetite.

12. The particle according to claim 11,
wherein the magnetic material contains the iron, and
wherein the magnetic material contains an iron atom at a content of 80% or more and 100% or less.

13. The particle according to claim 11,
wherein the magnetic material contains the nickel, and
wherein the magnetic material contains a nickel atom at a content of 80% or more and 100% or less.

14. The particle according to claim 1, wherein the functional group capable of binding a ligand is at least one kind selected from the group consisting of: a carboxyl group; an amino group; a thiol group; an epoxy group; a maleimide group; and a succinimidyl group.

15. The particle according to claim 14, wherein the functional group capable of binding a ligand is the carboxyl group.

16. The particle according to claim 1, wherein the resin has a siloxane bond.

17. An affinity particle comprising the particle of claim 1 and a ligand that binds to the particle.

18. The affinity particle according to claim 17, wherein the ligand is any one of an antibody and an antigen.

19. A test reagent comprising the affinity particle of claim 17 and a dispersion medium for dispersing the affinity particle.

20. A method of detecting a substance to be measured contained in a specimen, the method comprising:
a first step of adding a specimen containing a substance to be measured and a test reagent containing an affinity particle and a dispersion medium for dispersing the affinity particle into a container having a first ligand immobilized on a lower side in a gravity direction;
a second step of applying a magnetic field so that the affinity particle binds to the first ligand via the substance to be measured; and
a third step of applying a magnetic field so that the affinity particle free from binding to the first ligand via the substance to be measured is separated away from the first ligand,
wherein the affinity particle includes a particle comprising a magnetic particle containing a magnetic material,
wherein the magnetic particle has a resin on a surface,
wherein the particle has a volume average particle diameter of 0.4 μm or more and 1.5 μm or less,
wherein the particle has a density of 5.1 g/cm³ or more and 10.0 g/cm³ or less, and
wherein the resin has a functional group capable of binding a ligand, and
further wherein the first ligand and the second ligand are each bound to the substance to be measured.

21. The detection method according to claim 20, wherein the first ligand and the second ligand are identical to each other.

22. The detection method according to claim 20, wherein the first ligand and the second ligand are different from each other.

23. The detection method according to claim 20 further comprising detecting a signal from the affinity particle bound to the first ligand via the substance to be measured.
